(19) United States
(12) Patent Application Publication
(10) Pub. No.: US 2015/0230785 A1
(43) Pub. Date: Aug. 20, 2015

(54) SURGICAL PIN RETRACTOR SYSTEM

(71) Applicant: THE LITTLE SANDOX, LLC, LAGUNA BEACH, CA (US)

(72) Inventors: JOHN JAMES DeVERE, JR., CANYON LAKE, CA (US); ALI HAKIM MESIWALA, CLAREMONT, CA (US)

(73) Assignee: THE LITTLE SANDBOX, LLC, LAGUNA BEACH, CA (US)

(21) Appl. No.: 14/336,998

(22) Filed: Jul. 21, 2014

Related U.S. Application Data
(60) Provisional application No. 61/942,205, filed on Feb. 20, 2014.

(51) Int. Cl. A61B 17/02; A61B 1/32; A61B 17/30; A61B 1/00

(52) U.S. Cl. CPC A61B 17/02; A61B 1/00066; A61B 1/32; A61B 1/00142; A61B 17/30

(57) ABSTRACT

A surgical retractor system comprising the operative combination of a retractor blade for retracting tissue and an anchoring pin interconnectable therewith. The anchoring pin is preferably formed as an elongate structure having an anchoring mechanism formed at the distal-most end thereof that is designed to become securely anchored within bone or soft tissue. The anchoring pin is positioned through a grommet formed upon the retractor blade such that the anchoring pin remains firmly in position while the retractor blade retracts surrounding tissue. To facilitate handling of the system, a handle mechanism is detachably fastenable to the retractor blade. Multiple retractor devices can be utilized to facilitate tissue distraction, and further may be selectively positioned to retract tissue in a cranial-caudal orientation for use in anterior cervical spine surgery.

### CROSS-REFERENCE TO RELATED APPLICATIONS

The present application claims priority to U.S. Provisional Patent Application Ser. No. 61/942,205, filed Feb. 20, 2014, entitled SURGICAL PIN RETRACTOR SYSTEM, all of the teachings of which are incorporated herein by reference.

### STATEMENT RE: FEDERALLY SPONSORED RESEARCH/DEVELOPMENT

Not Applicable

### BACKGROUND

The present invention is directed to surgical retractor systems, and more particularly, retractor systems operative to remain securely anchored in bone or other tissue masses with a pin-type anchor and assume a free-standing configuration whereby an interconnected retractor blade retracts tissue about a surgical site.

Surgical retractors for retracting tissue about a surgical site are well-known in the art. In this regard, numerous types of retractor systems may be deployed in various types of specific surgical procedures, including orthopedic and neurological surgery such as anterior cervical disc surgery. Exemplary of such systems include those disclosed in pending U.S. patent application Ser. No. 13/445,571 filed Apr. 12, 2012 entitled ANTERIOR CERVICAL RETRACTOR SYSTEM; U.S. patent application Ser. No. 13/295,500 filed Nov. 14, 2011 entitled CERVICAL SPINE RETRACTOR; U.S. Pat. No. 8,118,737 B2, issued Feb. 12, 2012 entitled RETRACTOR DEVICE FOR CERVICAL SPINAL FUSION; U.S. Pat. No. 7,918,792 B2 issued Apr. 5, 2011 entitled SURGICAL RETRACTOR FOR USE WITH MINIMALLY INVASIVE SPINAL STABILIZATION SYSTEMS AND METHOD OF MINIMALLY INVASIVE SURGERY; U.S. Pat. No. 7,494,463 B2 issued Feb. 24, 2009 entitled RETRACTOR AND DISTRACTOR SYSTEM FOR USE IN ANTERIOR CERVICAL DISC SURGERY; and U.S. Pat. No. 5,944,658, issued Aug. 31, 1999 entitled LUMBAR SPINAL FUSION RETRACTOR AND DISTRACTOR SYSTEM, the teachings of all of which are expressly incorporated herein by reference. Such retractor systems are essential in order to provide the surgeon with access to a surgical site, as well as provide the surgeon with the necessary field of view to perform the surgical procedure. The retracted tissue also enables the surgeon to visually observe any problems that may arise during surgery and/or enable the surgery to take corrective measures as may be necessary.

Despite the need for the retraction of tissue during surgery, however, most retractor systems currently in use suffer from numerous drawbacks. For example, many retractor systems are mounted upon a bulky mechanical frame that forms a barrier about the periphery of the surgical site where the tissue is retracted. Such frame-based retractor systems often times hinder the surgeon's ability to access the surgical site and can further obstruct the surgeon's field of view given the bulkiness of the retractor frame structure.

Similarly, in other surgical retractor systems, the retractors must be interconnected with an external, stable structure, such as a bed frame and the like, that essentially requires the retractor system to be interconnected across the patient's body during surgery. Such retractor systems are cumbersome and can likewise interfere with a surgeon's ability to access a surgical site.

Additionally problematic with prior art surgical retractor systems is the inability of such systems to remain stable and firmly retract the tissue the system is designed to retract. Such retractor systems typically fail by not remaining secure in position and thus can become dislodged or otherwise cause the tissue sought to be retracted to encroach upon and/or impede the surgical site, as well as the surgeon's field of view.

Further drawbacks exist with prior art surgical systems insofar as the ability of such systems to remain anchored into position and retract tissue about a surgical site is dependent upon the device being secured into anatomical structures that are sub-optimal from the surgeon's perspective. With respect to the latter, and in the context of anterior cervical disc surgery, numerous surgical retractor systems are operative to remain anchored on opposed sides of a specific vertebrae, as opposed to retraction of tissue in a cranial-caudal orientation. In this regard, it would be substantially more advantageous to retract tissue via a retractor system that retracts tissue in a cranial orientation, on one hand, and a caudal orientation on the other hand. To do so would make it far easier for the surgeon to access the anterior cervical region, and would likewise substantially reduce the risk that the retractor system would interfere with the surgeon during a surgical procedure.

There is thus a substantial need in the art for a surgical retractor system that is operative to remain firmly secured into position and effectively retract tissue about a surgical site that is easy to deploy, becomes substantially secure into position, does not require a frame-type structure or any other structure external to the body for support, and can further be utilized with other like retractor systems to distract tissue in any of a variety of ways to thus provide substantially greater flexibility than prior art systems. There is further need in the art that is of simple construction and can further be secured into position such that tissue is retracted in a desired orientation, such as retraction via a cranial-caudal orientation, that has not hereto-for been available via prior art surgical retractor systems.

### BRIEF SUMMARY

The present invention specifically addresses and alleviates the above-identified deficiencies in the art. In this regard, the present invention is directed to a surgical retractor system that is operative to securely retract tissue about a surgical sight, especially for orthopedic or neurological surgery. The system is capable of retracting tissue in a manner that provides far greater flexibility as to where the retractor system may be placed into position within the human body, remains far more securely seated in position compared to prior art retractor devices, and can be paired or utilized with multiple like devices to selectively distract tissue to a greater degree than prior art retractor systems.

According to a preferred embodiment, the retractor system comprises the combination of a retractor blade having an anchoring pin interconnectable therewith. The retractor blade preferably comprises an elongate blade member having a proximal end, an intermediate portion, and distal end, the latter being operative to advance into and retract tissue. The proximal-most end of the blade member can optionally interconnect with a detachable handle mechanism, the latter being operative to assist handling and positioning of the retractor system.

The anchoring pin operatively engageable with the blade member enables the retractor system to remain firmly secured in position. In this regard, the elongate anchoring pin is insertable through a grommet formed upon the retractor blade such that the distal-most end of the anchoring pin extends therethrough. To selectively control the distance by which the elongate anchoring pin extends through the grommet formed on the retractor blade an abutment mechanism may be formed that cooperatively engages with the grommet to thus limit the length the distal end of the anchoring pin extends relative the distal-most end of the retractor blade.

Formed on the distal-most end of the anchoring pin is an anchoring mechanism that is operative to secure the anchoring pin into bone or soft tissue. According to a preferred embodiment, the distal-most end of the anchoring pin will be fashioned as a threaded screw that is operative to drill into a bone mass. To facilitate the ability of the anchoring pin to screw into or become embedded within such mass, it is contemplated that the proximal end of the anchoring pin may be fashioned as a bolt, nut or other similar structure such that when rotated, the anchoring mechanism formed on the distal-most end thereof correspondingly becomes anchored (e.g., screwed into position) into bone or another tissue site.

In use, the retractor system is utilized such that a incision is made and the retractor system, and in particular the distal-most end of the retractor blade, is inserted there-into. To facilitate handling of the system, the handle mechanism detachably fastenable to the proximal end of the blade member may be provided. Thereafter, the distal-most end of the anchoring pin is axially inserted through the grommet formed on the intermediate portion of the blade member and is anchored into position within a target site of bone. To that end, the anchoring pin will be manipulated (i.e., rotated) to cause the anchoring mechanism to become fixed.

Once anchored, the retractor system will remain resident with the blade member retracting the tissue in the desired manner until the surgical procedure is completed. With respect to removing the retractor system, the anchor mechanism is simply removed from the body mass into which the same is positioned, which may be accomplished by counter-rotation of the proximal end of the anchor pin and the retractor system subsequently removed.

In further refinements of the invention, the retractor system may include a light source, which may be formed upon the retractor blade or handle mechanism optionally attachable thereto, so as to provide an illumination source during surgery. The retractor system may also be fabricated from either disposable or reusable parts/materials. The retractor blade may also be formed to have a variety of shapes and configurations as may be desired for a specific procedure whereby a specific tissue site is to be retracted, so as to access a particular site. Moreover, the anchor mechanism formed upon the anchoring pin may be specifically configured to embed within a particular bone or, alternatively, may be specifically configured to embed within another tissue mass, such as soft tissue and the like, to facilitate retraction at other surgical sites and the like.

Along those lines, the retractor system of the present invention is believed to be particularly well suited for all types of orthopedic or neurologic surgery, and in particular anterior surgical spine surgery. In this regard, it is believed two or more retractor systems may be deployed in a manner that enables sufficient distraction to be achieved whereby respective ones or a pair of retractor systems are selectively anchored within the vertebrae to thus define an opening for performing surgery. Advantageously, and unlike prior art mechanisms, the retractor systems of the present invention can be deployed in a cranial-caudal configuration such that no retractor mechanism is positioned by or otherwise obstructing either side of the patient's neck where the surgery is performed.

Numerous other applications and surgical procedures where the retractor systems of the present invention may be deployed will be readily appreciated by those skilled in the art. In any such application, the simplistic and space-efficient design, ease of use, highly secure manner by which the retractor systems are secured into bone or otherwise retain their position, and the minimal impedance such retractor systems have in and about the surgical field created by such retracted tissue are some of the tremendous advantages not heretofore available to those skilled in the art.

### BRIEF DESCRIPTION OF THE DRAWINGS

These as well as other features of the present invention will become more apparent upon reference to the drawings.

FIG. 1 is a side, partially sagittal view of the neck and anterior cervical region of a surgical patient assuming a supine position.

FIG. 2 is the partial sagittal view of FIG. 1 showing a surgical retractor system constructed in accordance with a preferred embodiment of the present invention as deployed to retract tissue from a cranial orientation, the retractor system including an anchoring pin being secured to the C-5 vertebrae.

FIG. 3 is the partial sagittal view of FIG. 2 showing a second surgical retractor as constructed in accordance with a preferred embodiment of the present invention shown retracting tissue in a caudal orientation, the retractor including an anchoring pin shown inserted into the C-6 vertebrae.

FIG. 4 is the partial sagittal view of FIG. 3 wherein the retractor mechanism are shown independently anchored into position and operative to cooperatively distract tissue to allow for surgical access.

FIG. 5 is a perspective, exploded view of a surgical retractor system as constructed in accordance with a preferred embodiment of the present invention.

### DETAILED DESCRIPTION

The detailed description set forth below is intended as a description of the presently preferred embodiment of the invention, and is not intended to represent the only form in which the present invention may be implemented or performed. The description sets forth the functions and sequences of steps for practicing the invention. It is to be understood, however, that the same or equivalent functions and sequences may be accomplished by different embodiments and that they are also intended to be encompassed within the scope of the invention.

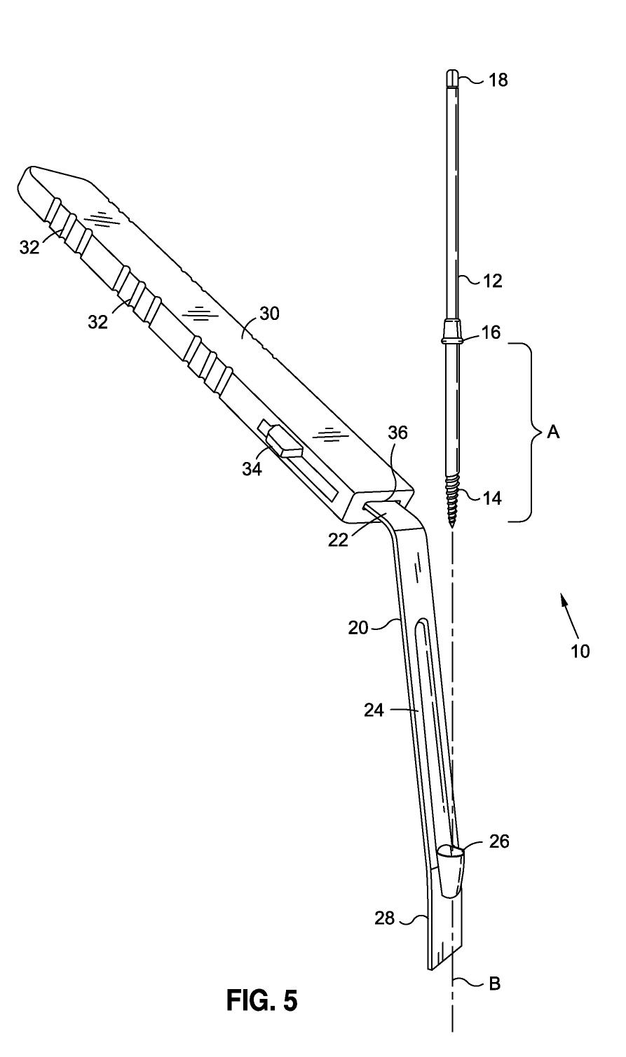

Referring now to the figures, initially to FIG. 5, there is illustrated surgical retractor system 10 as constructed in accordance with a preferred embodiment of the present invention. As shown, the system 10 comprises the operative combination of an anchoring pin 12, a retractor blade 20 and optionally, detachable handle 30, that enable the retractor system 10 to be selectively and securely anchored into position, via anchoring pin 12, such that retractor blade 20 can facilitate the retraction of tissue at a site selectively chosen by the surgeon.

With respect to anchoring pin 12, the same is preferably formed as an elongate member having a proximal end and a distal end. Formed on the distal-most end of anchoring pin 12 is an anchor mechanism 14 that is operative to become securely imbedded or fixed into position within bone or another target tissue mass. With respect to anchoring mechanism 14, the same may take any of a variety of forms well-known in the art, including the threaded screw embodiment as shown. As will be appreciated by those skilled in the art, the threaded screw as illustrated in FIG. 5 is operative to be drilled into a bone mass to thus enable the anchoring pin 12 and the retractor blade 20 interconnected therewith, discussed more fully below, to be securely fixed into position. To enable the anchoring mechanism 14 to be drilled or otherwise screwed into a bone or tissue mass, it is contemplated that the proximal-most end 18 of the anchoring pin 12 may be formed as a bolt, nut, or other polygonal shape to thus enable the anchoring pin 12 to be rotated via a ratchet or other like mechanism well-known in the art to thus correspondingly cause the anchoring mechanism 14 to drill into and become fixed into position within bone.

Formed along the intermediate portion of pin 12 is a cap or bell member 16 that is selectively formed a fixed distance “A” from the distal-most end 14 of the anchoring pin 12 to thus limit the length by which the anchoring mechanism 14 extends past retractor blade 20 when the anchoring pin 12 is interconnected therewith.

With respect to the interconnection between the anchoring pin 12 and retractor blade 20, FIG. 5 illustrates how the anchoring pin 12 interconnects with retractor blade 20. As illustrated, retractor blade 20 is provided with a proximal-most end 22, an intermediate segment 24, and distal-most end 28 as defined by arcs or bends formed along the length of the retractor blade 20. Formed at the juncture between intermediate portion 24 and distal-most end 28 of retractor blade 20 is a grommet 26 defining an aperture therethrough for receiving the distal-most portion of anchoring pin 12. As further illustrated, the cap or bell 16 formed upon the anchoring pin 12 is operative to abut with grommet 26 to thus limit the distance the distal-most portion of anchoring pin 12 extends through grommet 26 along axis “B,” and distally beyond distal-most end 28 of retractor blade 20.

As will be readily appreciated by those skilled in the art, the size and dimensions of anchoring pin 12 and retractor blade 20 may be adjusted as may be desired for a particular type of surgical procedure. In this regard, it is contemplated that the length of the anchoring pin 12, the type of anchoring mechanism 14 formed upon the distal-most end or anchoring pin 12, the length and width of retractor blade 20, and more particularly the distal-most end 28 thereof, may all be selectively sized and configured so as to anchor at a particular site of bone or tissue, in the case of anchoring pin 12, and retract a certain type and size of tissue mass, per retractor blade 20. Along those lines, it is contemplated that the anchoring pin 12, retractor blade 20 and optional handle 30 may all be fabricated from materials well-suited for use in surgical/surgical retractor applications and may include sterilizable materials or may be designed to either be disposable after single use or re-sterilizable for repeated use.

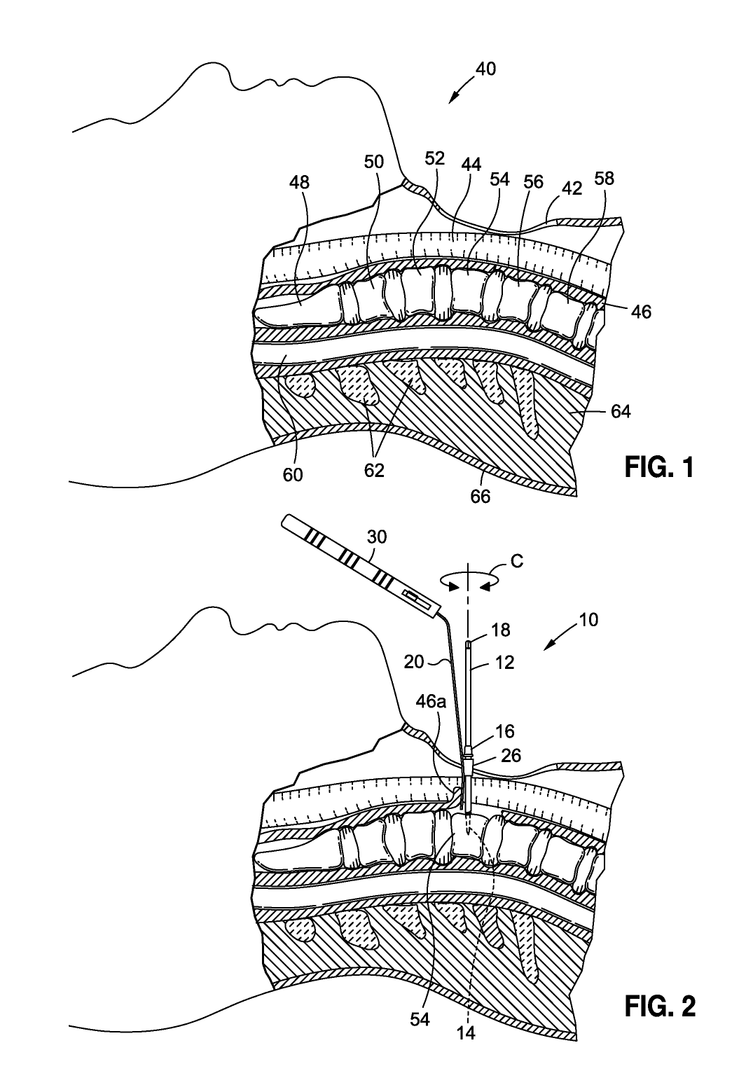

To facilitate the ability of the surgeon to secure the retractor system 10 into position, and more particularly the anchoring pin 12 thereof and thereafter manipulate the surgical blade 20 to retract tissue in the desired manner, it is contemplated that the system 10 will include a detachable handle 30 that is provided with a grippable or textured surface 32 and include a switch mechanism 34 that is operative to detachably interconnect with the proximal-most portion 22 of blade 20 via aperture 36. In this regard, in the embodiment shown, the distal-most end 28, intermediate portion 24 and proximal end 22 of the retractor blade 20 are shown to angle outwardly such that handle mechanism 30 extends at an acute angle away from anchoring pin 12 when axially aligned with axis “B.” As will be appreciated by those skilled in the art, the handle mechanism 30 enables the retractor blade 20 to extend through an incision to a site to which retraction is desired, and thereafter anchoring pin 12 extending through grommet 26 along axis “B” and secured into position, as will be more apparent with respect to FIGS. 1-4 discussed more fully below. In further refinements of the present invention, it is contemplated that handle mechanism 30 may be provided with other features to enhance the operability of such mechanism 30. For example, it is contemplated that a light may be incorporated as part of the handle mechanism 30, to thus provide a source of illumination onto the surgical site as may be desired.

As will be readily understood by those skilled in the art, the retractor system 10 of the present invention may find wide spread use in a number of orthopedic and neurologic applications, and is particularly well-suited whenever retraction of tissue is necessary during a surgical procedure where a target site of bone may serve as an anchoring point about which tissue can be retracted. As should be understood, however, that although the anchoring mechanism 14 as shown with the threaded embodiment may be ideally suited for anchoring into bone, it is contemplated that other anchoring mechanisms are contemplated that may be adapted to secure into soft tissue and the like in case of a procedure where it is desired to retract tissue about such an anchoring site.

Notwithstanding the fact that the anchoring system 10 of the present invention may be well suited for a wide variety of surgical applications, it is believed that the retractor system 10 may be particularly well suited for anterior cervical surgery, as illustrated in FIGS. 1-4. With respect to the first of such figures, FIG. 1 illustrates the head and neck of a patient 40, with a partial sagittal view of the anterior cervical region. As illustrated, an incision 42 formed on the neck of patient 40 creates surgical access to the structures therebeneath, including the esophagus 44, fascia 46, vertebrae 48-58 corresponding to vertebrae C2-7, spinal cord 60, spinous processes 62, neck muscles (i.e., trapezius) 64 and skin 66. As will be appreciated by those skilled in the art, the patient 40 would operatively assume such position when undergoing many of a variety of head and neck surgeries, and in particular anterior cervical disc surgery.

Referring now to FIG. 2, there is shown the surgical retractor system 10 as deployed to retract tissue in the patient depicted in FIG. 1. As illustrated, the system 10 is advanced into the surgical site such that anchoring pin 12 is received through grommet 26 such that the anchoring mechanism 14 of the distal-most end thereof becomes imbedded into the vertebrae 54. To facilitate the anchoring, the proximal end 18 of the anchoring pin 12 is rotated in the manner indicated by the letter “C” to facilitate not only the anchoring of the anchoring mechanism 14 into the vertebrae 54, but also removal thereof following the surgical procedure, discussed more fully below. As further illustrated, due to the interconnection between cap 16 and grommet 26, the depth by which the distal end of anchoring pin 12 extends past retractor blade 20 and into the bone 54 is selectively determined to have a fixed length so that the surgeon need not have to estimate the relative positions of the anchoring pin 12 and the retractor blade 20.

To facilitate the handling of the system 10, handle 30 may be manually manipulated by the surgeon such that the distal-most end of the retractor blade 20 is operative to retract tissue, in this case connective tissue flap 46a away from the point at which the retractor system 10 is anchored via anchoring pin 12 to thus enable the surgeon to have access to the site as desired. Along those lines, because the anchoring system 10 is a free-standing system that does not require any type of frame mechanism or require any type of attachment to any external structure, the retractor system 10 may be deployed in any manner that the surgeon sees fit so long as a secure anchoring point to which the anchoring pin 12 can be affixed can be accessed.

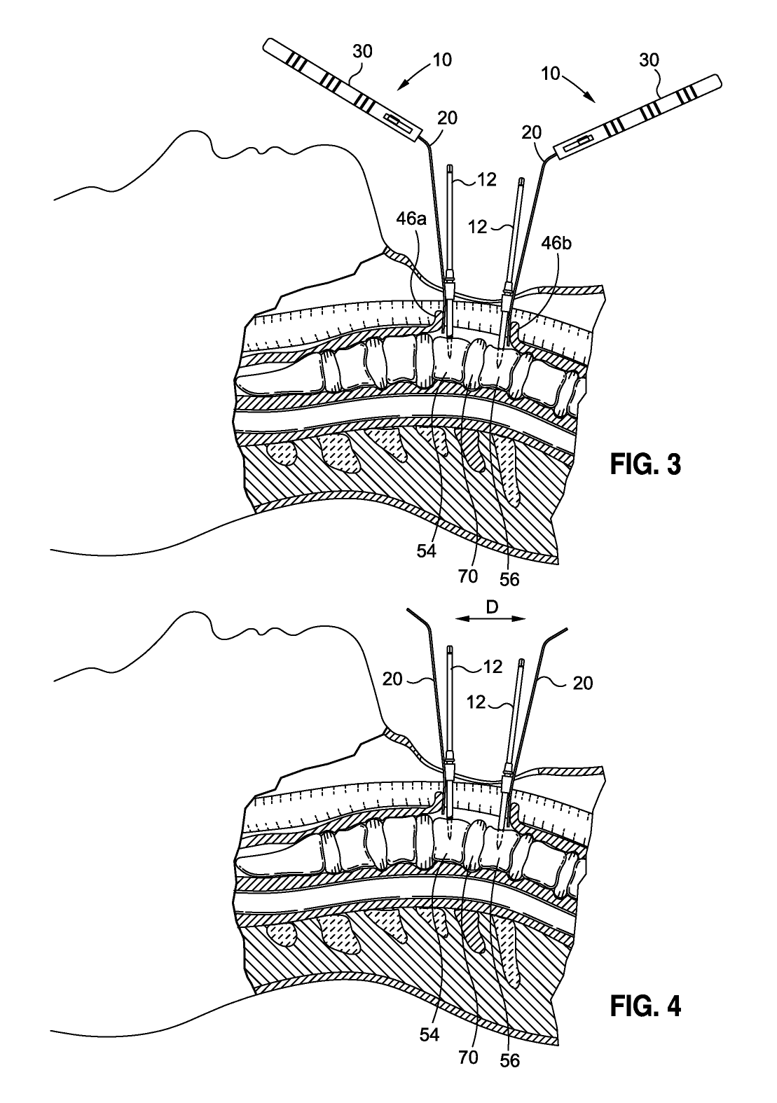

Referring now to FIG. 3, there is shown a combination of retractor systems 10 as utilized to cooperatively retract tissue about a surgical site in the anterior cervical spine region. As illustrated, the first of such retractor systems is shown retaining tissue in a cranial-type orientation, whereas the second system is shown retracting tissue in a caudal-type configuration. With respect to the latter, anchoring pin 12 is shown anchored into vertebrae 56 with the retractor blade 20 retracting tissue 46b in a caudal-oriented manner thus exposing disc 70 as may be required or desired for a particular surgical procedure. As discussed above, each retractor system 10 is free-standing in nature and operative to act as a stand-alone point about which tissue may be retracted. Accordingly, it is believed that two or more of such systems, as depicted in FIG. 3, may be deployed so as to selectively create an opening of retracted tissue as may be desired for any of a variety of surgical procedures, and in particular neurological and/or orthopedic procedures where retraction of tissue about a anchoring point in bone will be deemed desirable.

As illustrated in FIG. 4, there is shown a potential use of the retractor systems 10 of the present invention for use in accessing the inter-cervical spine region whereby the anchoring systems define a surgical field “D” by virtue of being anchored in position in a cranial-caudal configuration. As will be appreciated by those skilled in the art, most conventional anchoring mechanisms, and in particular those used for inter-cervical disc surgery, do not allow for the retraction of tissue in a cranial-caudal configuration but rather rely upon bulky frame structures that retract tissue from side to side using external structures as points to which the retractor mechanisms are attached. The present systems advantageously dispense with all such requirements and can be deployed in the aforementioned manner at any location as deemed desirable by the physician. As such, so long as a point of attachment can be located and a retractor blade 20 selectively positioned so as to retract tissue in a desired manner, the systems 10 of the present invention may find wide spread applicability for of a wide variety of surgical procedures.

Additional modifications and improvements of the present invention may also be apparent to those of ordinary skill in the art. Thus, the particular combination of parts and steps described and illustrated herein is intended to represent only certain embodiments of the present invention, and is not intended to serve as limitations of alternative devices and methods within the spirit and scope of the invention.

What is claimed is:

1. A surgical retractor system comprising:
an elongate anchoring pin having a proximal end, an intermediate portion and a distal end;
an anchoring mechanism formed upon said distal end of said anchoring pin, said anchoring mechanism being operative to become detachably imbedded within a selected target site of bone or soft tissue; and
an elongate retractor blade having a proximal end, an intermediate portion, and a distal end, said retractor blade having a grommet formed thereon and defining an aperture therethrough for receiving and interconnecting with said anchoring pin such that said distal end of said anchoring pin is extensible through said aperture of said grommet and operative to become imbedded within said selected target site of bone or soft tissue.

2. The anchoring system of claim 1 further comprising a handle mechanism detachably fastenable to said proximal end of said retractor blade.

3. The anchoring system of claim 1 wherein said retractor blade has a first bend formed thereon defining a transition from said proximal end to said intermediate portion adjacent said proximal end and a second bend defining a transition from said distal end to said intermediate portion adjacent thereto.

4. The anchoring system of claim 3 wherein said grommet is formed upon said second bend defining said transition from said distal portion of said retractor blade to said intermediate portion of said retractor blade.

5. The surgical retractor system of claim 1 wherein said anchoring pin has a cap portion formed intermediate said distal and proximal ends for engaging with said grommet formed upon said retractor blade, said engagement between said cap member and said grommet limiting the length by which the distal end of said anchoring pin extends through said aperture defined by said grommet.

6. The retractor system of claim 1 wherein said anchoring pin and retractor blade are formed from a sterilizable material.

7. The retractor system of claim 1 wherein said anchoring mechanism comprises a threaded screw operative to drill into bone.

8. The retractor system of claim 7 wherein said proximal end of said anchoring pin is fashioned as a bolt to facilitate the ability of the anchoring mechanism to drill into bone and be removed therefrom.

9. A surgical retractor system consisting of:
an elongate anchoring pin having a distal end with an anchor mechanism formed thereon for securing said anchoring pin to bone, said anchoring pin having a proximal end with a bolt member formed thereon for facilitating the ability of the anchoring device to become imbedded within bone; and
a retractor blade having a proximal end, an intermediate portion, and distal end, said retractor blade being engageable with said anchoring pin and operative to retract tissue when said anchoring mechanism formed on said anchoring pin is imbedded in bone.

10. A surgical retractor system comprising:
an elongate retractor blade having a proximal end, and intermediate portion and a distal end;
a handle member detachably fastenable to said proximal end of said retractor blade;
an elongate anchoring pin having a proximal end, intermediate portion and distal end, an anchoring mechanism being formed upon said distal end of said anchoring pin for imbedding into bone; and
an interconnection means for interconnecting said anchoring pin with said retractor blade, said interconnection means being operative to position said distal end of said retractor blade in a tissue retraction mode when said anchoring mechanism formed on said distal end of said anchoring pin is imbedded in bone.
